(19) United States
(12) Patent Application Publication
(10) Pub. No.: US 2025/0380788 A1
(43) Pub. Date: Dec. 18, 2025

(54) APPARATUSES AND METHODS FOR SUNSCREEN APPLICATION

(71) Applicant: Aubry Cook, Provo, UT (US)

(72) Inventor: Aubry Cook, Provo, UT (US)

(21) Appl. No.: 18/746,885

(22) Filed: Jun. 18, 2024

(51) Int. Cl. A45D 34/04; A61K 8/29; A61K 8/92; A61Q 17/04

(52) U.S. Cl. CPC A45D 34/04; A61K 8/29; A61K 8/922; A61Q 17/04

(57) ABSTRACT

Liquid applicator devices and related methods are disclosed. A liquid applicator device includes a liquid container having a first end and a second end, a sunscreen mixture within the liquid container, and a tip configured to couple to the first end. The sunscreen mixture includes a pigment agent, a thinning agent, and a sunscreen material. The sunscreen mixture has a viscosity of less than or equal to 100 centapoise (cP). The tip is configured to release the sunscreen mixture on a surface in contact with the tip.

### BACKGROUND

Cosmetics, such as sunscreens, include mixtures of materials that are applied to cover a portion of a human skin. For example, sunscreens provide protection of the covered portion of the skin. To wear conventional liquid-type sunscreens, such sunscreens are applied using spray bottles, bottles or tubes with either spray nozzles, dropper tips, pad applicators and/or sponge wedges to apply the liquid sunscreen evenly on the skin. However, the conventional sunscreens' receptacles and applicators, such as spray bottles, dropper tips, pad applicators and/or sponge wedges, are generally designed to evenly apply the sunscreen over a relatively large surface of skin and may not allow a user to apply a liquid-type sunscreen with precision.

### BRIEF DESCRIPTION OF THE DRAWINGS

One or more embodiments of the disclosure will now be described, by way of example only, with reference to the accompanying drawings.

FIG. 1 illustrates a method of providing a sunscreen mixture to a liquid container of a liquid applicator device in according to some embodiments.

FIG. 2 is an exploded side view of a liquid applicator device in accordance with examples described herein.

FIG. 3A is a perspective view of a tip of a liquid applicator device in accordance with examples described herein.

FIG. 3B is a side view of a tip of a liquid applicator device in accordance with examples described herein.

FIG. 3C is a front view of a nib of a liquid applicator device in accordance with examples described herein.

FIG. 4A is a top view of a portion of a liquid applicator device and a stroke by the liquid applicator device in accordance with examples described herein.

FIG. 4B is a side view of a portion of a liquid applicator device and a stroke by the liquid applicator device in accordance with examples described herein.

FIG. 5A is a schematic diagram of a liquid applicator device in accordance with examples described herein.

FIG. 5B to FIG. 5D are schematic diagrams of tips in accordance with examples described herein.

FIG. 6A is an exploded side view of a liquid applicator device in accordance with examples described herein.

FIG. 6B is a side view of a nib of a liquid applicator device in accordance with examples described herein.

FIG. 6C is a perspective view of a nib of a liquid applicator device in accordance with examples described herein.

FIGS. 7A and 7B are side views of liquid refill devices in accordance with examples described herein.

FIG. 8 is a side view of a liquid refill device in accordance with examples described herein.

### DETAILED DESCRIPTION

In the following detailed description, reference is made to the accompanying drawings, which form a part hereof, and in which are shown, by way of illustration, specific examples of embodiments in which the present disclosure may be practiced. These embodiments are described in sufficient detail to enable a person of ordinary skill in the art to practice the present disclosure. However, other embodiments enabled herein may be utilized, and structural, material, and process changes may be made without departing from the scope of the disclosure.

The illustrations presented herein are not meant to be actual views of any particular method, system, device, or structure, but are merely idealized representations that are employed to describe the embodiments of the present disclosure. In some instances, similar structures or components in the various drawings may retain the same or similar numbering for the convenience of the reader; however, the similarity in numbering does not necessarily mean that the structures or components are identical in size, composition, configuration, or any other property.

The following description may include examples to help enable one of ordinary skill in the art to practice the disclosed embodiments. The use of the terms “exemplary,” “by example,” and “for example,” means that the related description is explanatory, and though the scope of the disclosure is intended to encompass the examples and legal equivalents, the use of such terms is not intended to limit the scope of an embodiment or this disclosure to the specified components, steps, features, functions, or the like.

It will be readily understood that the components of the embodiments as generally described herein and illustrated in the drawings could be arranged and designed in a wide variety of different configurations. Thus, the following description of various embodiments is not intended to limit the scope of the present disclosure, but is merely representative of various embodiments. While the various aspects of the embodiments may be presented in the drawings, the drawings are not necessarily drawn to scale unless specifically indicated.

Furthermore, specific implementations shown and described are only examples and should not be construed as the only way to implement the present disclosure unless specified otherwise herein. Elements and functions may be shown in block diagram form in order not to obscure the present disclosure in unnecessary detail. Conversely, specific implementations shown and described are exemplary only and should not be construed as the only way to implement the present disclosure unless specified otherwise herein. Additionally, block definitions and partitioning of logic between various blocks is exemplary of a specific implementation. It will be readily apparent to one of ordinary skill in the art that the present disclosure may be practiced by numerous other partitioning solutions. For the most part, details concerning timing considerations and the like have been omitted where such details are not necessary to obtain a complete understanding of the present disclosure and are within the abilities of persons of ordinary skill in the relevant art.

Any reference to an element herein using a designation such as “first,” “second,” and so forth does not limit the quantity or order of those elements, unless such limitation is explicitly stated. Rather, these designations may be used herein as a convenient method of distinguishing between two or more elements or instances of an element. Thus, a reference to first and second elements does not mean that only two elements may be employed there or that the first element must precede the second element in some manner. In addition, unless stated otherwise, a set of elements may include one or more elements.

As used herein, the term “substantially” in reference to a given parameter, property, or condition means and includes to a degree that one of ordinary skill in the art would understand that the given parameter, property, or condition is met with a small degree of variance, such as, for example, within acceptable manufacturing tolerances. By way of example, depending on the particular parameter, property, or condition that is substantially met, the parameter, property, or condition may be at least 90% met, at least 95% met, or even at least 99% met.

As used herein, the term “sunscreen material” refers to chemical or mineral sunscreens that absorb, block, or reflect ultraviolet (UV) radiation. A sunscreen material may include chemical sunscreens such as avobenzone, octocrylene, octinoxate, or oxybenzone, or physical (e.g., mineral) sunscreens such as zinc oxide. A sunscreen material may have a higher sun protection factor (SPF) than other ingredients in a sunscreen mixture than a thinning agent (e.g., an SPF oil) or a pigment agent, although one or more of the thinning agent or the pigment agent may also have sunscreen properties.

Viscosity of liquids is discussed herein. It will be apparent to a person of ordinary skill in the art that viscosity of a liquid may change responsive to changes in temperature and/or pressure. For purposes of this disclosure, values and ranges for liquid viscosity are given for a temperature substantially at room temperature (e.g., between 20 and 25 degrees Celsius, or equivalently between 68 and 77 degrees Fahrenheit). Although changes in pressure may change a viscosity of a liquid, it will be apparent to those of ordinary skill in the art that changes in viscosity due to changes in pressure (e.g., due to elevation changes) in the open atmosphere may be negligible compared to changes in viscosity due to temperature changes.

Sunscreen products available on the market generally fit into categories including spray sunscreens, lotion sunscreens, and stick applicators. The viscosity of liquid sunscreens (e.g., spray sunscreens and lotion sunscreens) may be selected at least partially based on the intended manner of application. For example, spray sunscreens may have relatively low viscosities (e.g., between 5 and 20 centipoise (cP) to allow the sunscreens to be dispensed effectively from aerosol containers and to provide uniform coverage of the product on a person's skin. By contrast, lotion sunscreens may have relatively high viscosities (e.g., between 1,000 to 10,000 cP) to allow the lotion sunscreens to be easily dispensed from a bottle and handled for even application to the skin. Stick applicators are generally filled with an almost solid sunscreen similar in texture and function to deodorant in a deodorant stick, and are often used for applying sunscreen to the skin of a person's face.

While these conventional sunscreen products may generally be designed with the goal of even application to a large area of skin, it may be desirable, in some instances, to apply sunscreen to only a limited, detailed portion of skin. For example, sunscreen art may involve selectively applying sunscreen to create pictures, writings, designs, or patterns to temporarily tan a negative of the applied pictures, writings, designs, or patterns on the skin responsive to exposing the skin to the sun. The effect may be similar to a temporary tattoo that does not wash off, but fades over time as the pigmentation of the tanned skin fades. The optimization of most available sunscreen products for applying the sunscreens evenly to large areas of skin, however, is not conducive to detailed application for sunscreen art.

Inks and certain types of paints, however, have viscosities that are more conducive than available liquid sunscreen products to detailed application to skin. For example, inks for various pens may have viscosities ranging anywhere from 1 to 100 cP, depending on the type of pen and the mechanism used for dispensing ink. Also, paints for paint markers may have viscosities ranging anywhere from about 10 to 100 cP.

Disclosed herein are dispensing apparatuses (e.g., sunscreen pens, sunscreen markers), sunscreen mixtures, and methods of mixing sunscreen mixtures for detailed application of sunscreen to skin. Embodiments disclosed herein may be used for sunscreen art or other forms of sunscreen application that would benefit from the ability to apply the sunscreen in detailed arrangements on the skin similarly to ink or paint. By way of non-limiting example, embodiments disclosed herein may use sunscreen mixtures having viscosities between 10 and 100 cP. Also by way of non-limiting example, embodiments disclosed herein may use sunscreen mixtures having viscosities between 20 and 100 cP. As a further non-limiting example, embodiments disclosed herein may use sunscreen mixtures having viscosities between 30 and 100 cP. Since these viscosity ranges generally have higher viscosities than viscosities of spray sunscreens and lower viscosities than viscosities of lotion sunscreen materials, some embodiments disclosed herein may involve thinning down a sunscreen material having a relatively high viscosity (e.g., between 1,000 and 10,000 cP) with a thinning agent to reduce the viscosity to less than 100 cP to enable the sunscreen mixture to be used in a sunscreen pen or marker. For example, various sun protection factor (SPF) oils (e.g., argan oil, raspberry seed oil, avocado oil, coconut oil, etc.) may be used to thin down the sunscreen mixture.

The application of sunscreen for sunscreen art may also benefit from a sunscreen mixture that allows a person that is applying the sunscreen mixture (e.g., a sunscreen artist) to visibly distinguish between areas of skin that have already had sunscreen applied thereto and areas of skin that have not had sunscreen applied thereto. Accordingly, sunscreen mixtures used by embodiments disclosed herein may include opaquely-colored chemicals such as titanium dioxide (TiO2) powder to give the sunscreen mixtures sufficient pigment to visibly distinguish areas of the skin that have had the sunscreen mixture applied thereto from areas that have not had the sunscreen mixture applied thereto. TiO2 is white-pigmented and acts as an ultraviolet (UV) light filter due to its UV absorption and scattering properties and may be used as a pigment agent for a sunscreen mixture.

According to various embodiments, a sunscreen mixture may include a sunscreen material (e.g., an off-the-shelf sunscreen lotion), a thinning agent, and a pigment agent. In some embodiments the sunscreen mixture may include equal parts (e.g., by volume) of the sunscreen material, the thinning agent, and the pigment agent.

Apparatuses and methods for sunscreen application with precision are also disclosed. An example apparatus may be a liquid applicator device that includes a tip including a nib, a liquid container and a tail. In some examples, the nib may have a first edge that provides a first stroke thickness and a second edge shorter than the first edge that provides a second stroke thickness less than the first stroke thickness. In some examples, the nib may further include a corner at a first end of the first edge and a second end of the second edge, where the corner provides a third stroke thickness less than the second stroke thickness.

Another example apparatus may be a liquid applicator device including a liquid container that may store a liquid and a tail that seals the liquid in the liquid container. The liquid container has a first end and a second end opposite to the first end. The tail may couple to the second end of the liquid container, and the first end of the liquid container may couple to a tip. In some examples, the liquid applicator device may further include a porous foam at the first end of the liquid container. The porous foam may absorb the liquid in the liquid container, and further releases the liquid to the tip evenly (e.g., at a constant speed across a touching surface). In some examples, the liquid applicator device may further include a liquid channel having an inner opening and an outer opening to be in contact with the porous foam, where a radius of the inner opening is smaller than a radius of the outer opening. In some examples, the liquid applicator device may further include one or more balls that may move in the liquid container to keep density of materials in the liquid even within the liquid container.

In some examples, the tip may include a nib having a first edge and a second edge shorter than the first edge. In some examples, the nib has a uniform radius that provides a uniform stroke thickness regardless of an angle and a direction of a stroke. In some examples, the tip may include a ballpoint to prevent hardening of the liquid at the tip.

In the following description, various embodiments will be described. For purposes of explanation, specific configurations and details are set forth in order to provide a thorough understanding of the embodiments. However, it will also be apparent to skilled persons that the embodiments may be practiced without the specific details. Furthermore, well-known features may be omitted or simplified in order not to obscure the embodiment being described.

While the following preferred embodiments refer specifically to examples of sunscreen mixture application, it should be appreciated that the same or similar apparatuses and methods may apply to other liquid application technology and no limitation is intended.

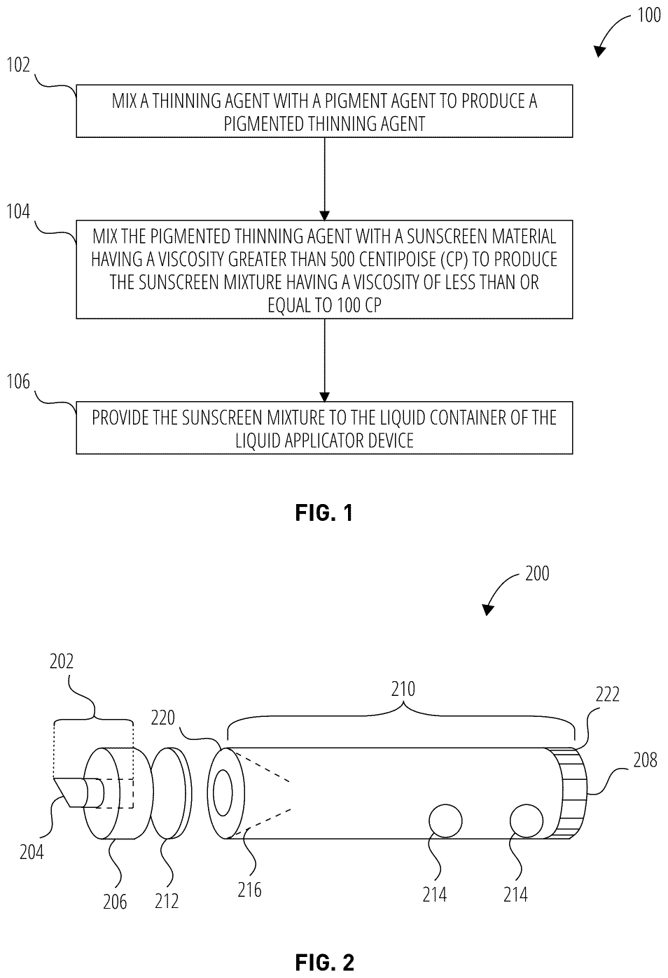

Referring to FIGS. 1A-8, exemplary embodiments of apparatuses and methods for applying sunscreen are disclosed. The reader should understand that the embodiments discussed herein may be implemented in many alternate forms and variations.

FIG. 1 is a flowchart illustrating a method 100 of providing a sunscreen mixture to a liquid container of a liquid applicator device. At operation 102, the method 100 includes mixing a thinning agent with a pigment agent to produce a pigmented thinning agent. The mixing may continue until there are no clumps and the pigmented thinning agent has a smooth texture. In some embodiments, the pigmented thinning agent may be sufficiently runny to cause the pigmented thinning agent to drip off of a mixing instrument (e.g., a spoon). Additional thinning agent may be added to the pigmented thinning agent until the smooth, runny texture is achieved.

In some embodiments, mixing the thinning agent with the pigment agent to produce the pigmented thinning agent includes mixing the thinning agent with titanium dioxide (TiO2) to produce the pigmented thinning agent. In some embodiments, mixing the thinning agent with the pigment agent to produce the pigmented thinning agent includes mixing a sun protection factor (SPF) oil with the pigment agent to produce the pigmented thinning agent. In some embodiments, the SPF oil includes one or more of raspberry seed oil, avocado oil, argan oil, soybean oil, olive oil, shea butter, almond oil, sesame seed oil, macadamia oil, hemp seed oil, jojoba oil, coconut oil, wheat germ oil, cottonseed oil, peanut oil, or sesame oil.

At operation 104, the method 100 includes mixing the pigmented thinning agent with a sunscreen material (e.g., an off-the-shelf sunscreen lotion) having a viscosity greater than 500 centipoise (cP) to produce the sunscreen mixture having a viscosity of less than or equal to 100 cP. In some embodiments, the viscosity of the sunscreen mixture is greater than 10 cP. In some embodiments, the viscosity of the sunscreen mixture is greater than 20 cP. In some embodiments, the viscosity of the sunscreen mixture is greater than 30 cP. In some embodiments, the sunscreen mixture is at least substantially equal parts, by volume, thinning agent, pigment agent, and sunscreen material.

At operation 106, the method 100 includes providing the sunscreen mixture to the liquid container of the liquid applicator device.

FIG. 2 is an exploded side view of a liquid applicator device 200 in accordance with examples described herein. The liquid applicator device 200 may include a tip 202 including a nib 204 and a tip adaptor 206, a tail 208 and a liquid container 210. The liquid container 210 may have an end 220 and another end 222 opposite to the end 220. The tip 202 may couple to the liquid container 210 at the end 220. The tip adaptor 206 attached to the nib 204 securely couples to the end 220 of the liquid container 210 (e.g., a fluid tight coupling). The tail 208 may couple to the liquid container 210 at the end 222 (e.g., also a fluid-tight coupling). In some embodiments, the liquid applicator device 200 may include a foam material 212 at the end 220 of the liquid container 210, between the liquid container 210 and the tip 202. The liquid applicator device 200 may include a liquid channel 216 at the end 220.

The liquid container 210 may store a liquid, such as a sunscreen mixture. The tail 208 may be coupled to the end 222 of the liquid container 210 to seal the liquid in the liquid container 210. In some examples, the foam material 212 at the end 220 of the liquid container 210 may be a porous foam that may absorb the liquid in the liquid container 210 and release the liquid to the tip 202 evenly (e.g., at an at least substantially constant speed across a touching surface). In some examples, a density of the foam material 212 may be relatively low (relatively high porosity), as compared to foam materials used in ink and paint pens, to prevent blobs and inconsistency. A lower density (e.g., a higher porosity) of the foam material 212 may provide for smooth release of the liquid including a sunscreen mixture through the nib 204.

The liquid channel 216 may have an inner opening and an outer opening to be in contact with the foam material 212. In some examples, an area of the inner opening is smaller than an area of the outer opening. The liquid channel 216 may hold the foam material 212 and allow the nib 204 to be pressed into the liquid applicator device 200 so that liquid is squeezed out through the nib 204. Pressing the nib 204 into the liquid applicator device 200 a few times may encourage the liquid to start seeping through the foam material 212 and the nib 204 to enable application of the liquid.

In some examples, the liquid applicator device 200 may further include one or more balls 214 that may move within the liquid container 210 to mix materials of the sunscreen mixture in the liquid container 210. The moving balls 214 may contribute to mixing a sunscreen mixture in the liquid container 210 responsive to shaking of the liquid applicator device 200.

In some examples, the liquid in the liquid container 210, such as the sunscreen mixture, may include a mixture of a thinning agent (e.g., an SPF oil), a pigment agent (e.g., TiO2), and one or more sunscreen materials (e.g., an off-the-shelf sunscreen lotion) that block ultraviolet rays of the sun. In some examples, a ratio of thinning agent, pigment agent, and sunscreen material may be at least substantially 1:1:1 by volume (e.g., at least substantially the same amount, in volume, of the thinning agent, the pigment agent (e.g., TiO2 powder), and the sunscreen material).

In some examples, the sunscreen material may include avobenzone, octinoxate or oxybenzone. In some examples, the sunscreen material may include titanium dioxide. In some examples, the pigment agent may include dry powdered titanium dioxide. In some examples, the thinning agent (e.g., an SPF oil) may include one any of raspberry seed oil (SPF 28-50), avocado oil (SPF 4-15), argan oil (SPF 15), soybean oil (SPF 10), olive oil (SPF 2-8), shea butter (SPF 3-6), almond oil (SPF 5), sesame seed oil (SPF 4), macadamia Oil (SPF 6), hemp seed oil (SPF 6), jojoba oil (SPF 4), coconut oil (SPF 2-8), wheat germ oil (SPF 20), cottonseed oil (SPF 6-10), peanut oil (SPF 4-10), and/or sesame oil (SPF 4-10).

For instance, argan oil and titanium dioxide may be mixed at a ratio of 1:1 and stirred well until the texture of the mixture becomes smooth without clumps. In some examples, more oil may be added to achieve a “runny texture” that drips off from a spoon. A sunscreen material of a similar amount to each of the argan oil or titanium dioxide may be added and thoroughly mixed. To obtain an appropriate “runny texture,” the ratio of the ingredients may be further adjusted. Once the mixture is prepared, the mixture may be stored in the liquid container 210. The balls 214 in the liquid container 210 may stir the active ingredients (e.g., when the liquid applicator device 200 is shaken) sufficiently for even application of the sunscreen for each use.

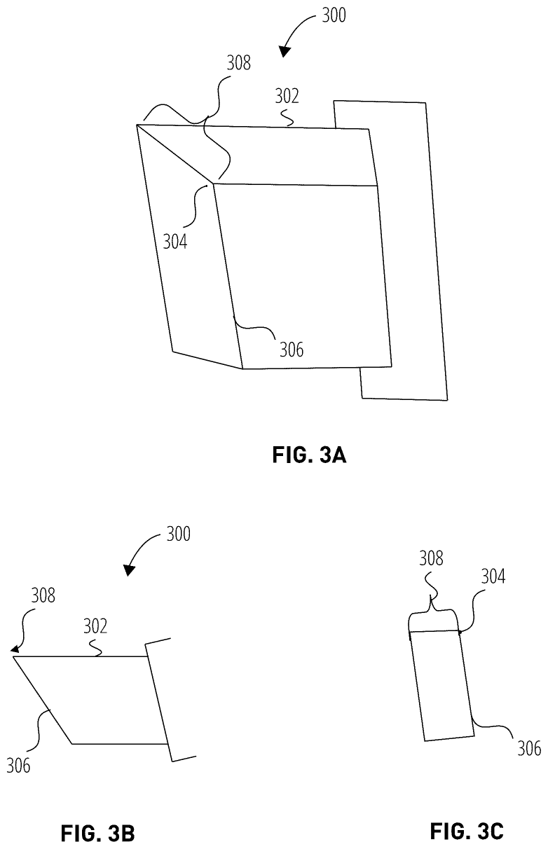

FIG. 3A is a perspective view of a tip 300 of a liquid applicator device in accordance with examples described herein. FIG. 3B is a side view of the tip 300 of a liquid applicator device in accordance with examples described herein. FIG. 3C is a front view of a nib 302 of a liquid applicator device in accordance with examples described herein. In some embodiments, the liquid applicator device may be the liquid applicator device 200, the tip 300 may be the tip 202, and the nib 302 may be the nib 204 of the liquid applicator device 200 in FIG. 2. The nib 302 may have a first edge 306 and a second edge 308. The first edge 306 and the second edge 308 may provide different stroke thicknesses. In some examples, a length of the second edge 308 may be shorter than a length of the first edge 306. Thus, the second edge 308 may provide a stroke thickness that is less than a stroke thickness of the first edge 306, caused by the shorter length of the second edge 308. In some examples, the nib 302 may further include a corner 304 that is at an end of the second edge 308 and an end of the first edge 306. The corner 304 may provide a stroke thickness that is less than the second stroke thickness. Using the corner 304, a pinpoint application of the liquid may be performed. In some examples, the first edge 306 may be angled. Based on a stroke of a user, the corner 304, the first edge 306, or the second edge 308 may be in contact with a surface, such as a skin of a human. In some examples, surfaces defined by the first edge 306 and the second edge 308 may be in contact with the surface. Thus, a precise application of the sunscreen may be performed using the corner 304 or the second edge 308, whereas a thicker efficient application of the sunscreen may be performed using the first edge 306.

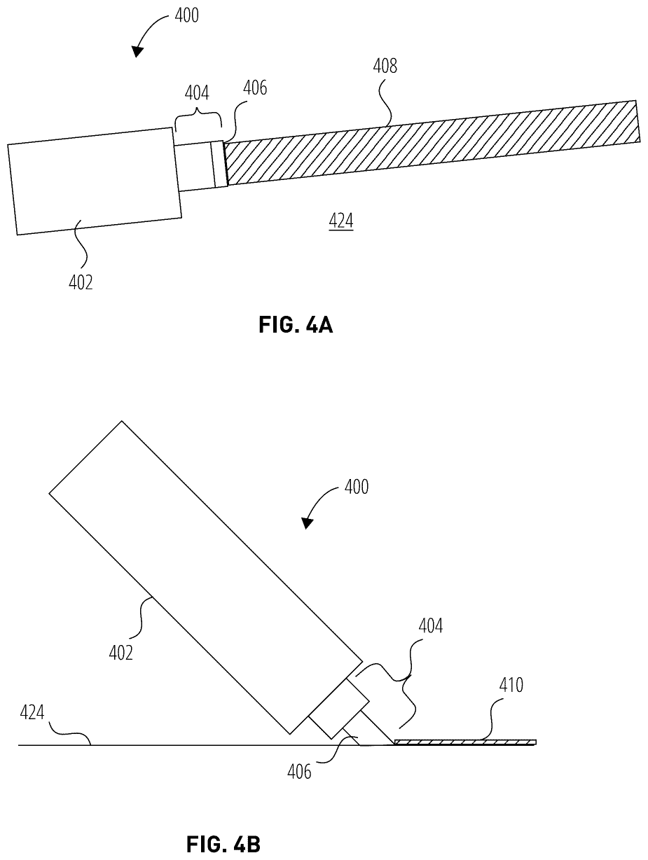

FIG. 4A is a top view of a portion of a liquid applicator device 400 and a stroke 408 by the liquid applicator device in accordance with examples described herein. In some examples, the liquid applicator device 400 may be the liquid applicator device 200 of FIG. 2. The portion of the liquid applicator device 400 may include a liquid container 402 and a tip 404 including a nib 406. In some embodiments, the tip 404 may be the tip 300 of FIGS. 2A-2C. The tip 404 may be in contact with a surface 424, such as a human skin at an edge, such as the first edge 306 with a stroke direction perpendicular to a length of the edge. Thus, a thick width of application of the sunscreen may be achieved by the thick stroke 408.

FIG. 4B is a side view of a portion of a liquid applicator device 400 and a stroke 410 by the liquid applicator device in accordance with examples described herein. The tip 404 may be in contact with a surface 424, such as a human skin, at an edge, such as the second edge 308, with a stroke direction in a direction of the edge. Thus, a thin application width of the sunscreen may be achieved by the thin stroke 410. Thus, a thickness of the liquid application may be controlled, and relatively precise application of the sunscreen may be achieved.

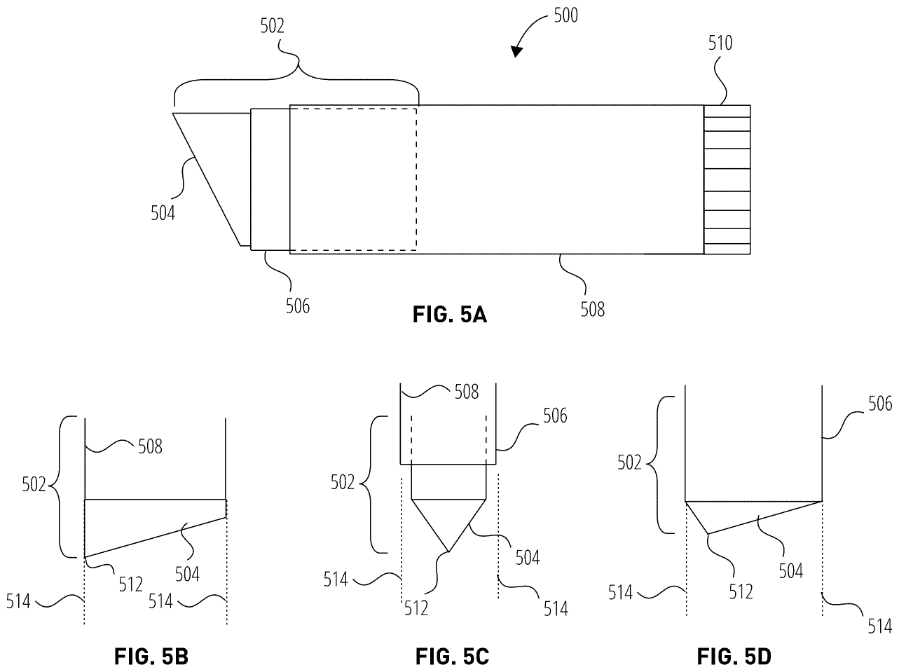

FIG. 5A is a schematic diagram of a liquid applicator device 500 in accordance with examples described herein. The liquid applicator device 500 may include a tip 502 including a nib 504 and a tip adaptor 506, a liquid container 508, and a tail 510. FIG. 5A is a side view of the liquid applicator device 500 in accordance with examples described herein. FIG. 5B to FIG. 5D are schematic diagrams of tips 502 in accordance with examples described herein.

In some examples, the nib 504 may have a point 512 that is at least substantially aligned with an outer surface of the liquid container 508 as shown in FIG. 5B (extension lines 514 shown in FIGS. 5B, 5C, and 5D extending past the outer surface of the liquid container 508). In some examples, the nib 504 may have a point 512 that is between extension lines 514 of opposite sides of the outer surface of the liquid container 508, as shown in FIGS. 5C and 5D. In some examples, the point 512 may be at least substantially at the same distance from the extension lines 514 of the opposite sides of the outer surface of the liquid container 508 as shown in FIG. 5C (e.g., the point 512 may be at least substantially centered between the extension lines 514). In some examples, the point 512 may be at the different distances from the extension lines 514 of the opposite sides of the outer surface of the liquid container 508 as shown in FIG. 5D (e.g., the point 512 may be off-center). The different nibs 504 illustrated in FIGS. 5B-5D may allow different thicknesses of lines to be drawn by the liquid applicator device 500. In some embodiments, the liquid applicator device 500 may be configured for swapping out the tip 502 such that multiple different tips 502 (e.g., similar to the tips 502 illustrated in FIGS. 5B-5D) may be changed according to a user's wishes and application goals.

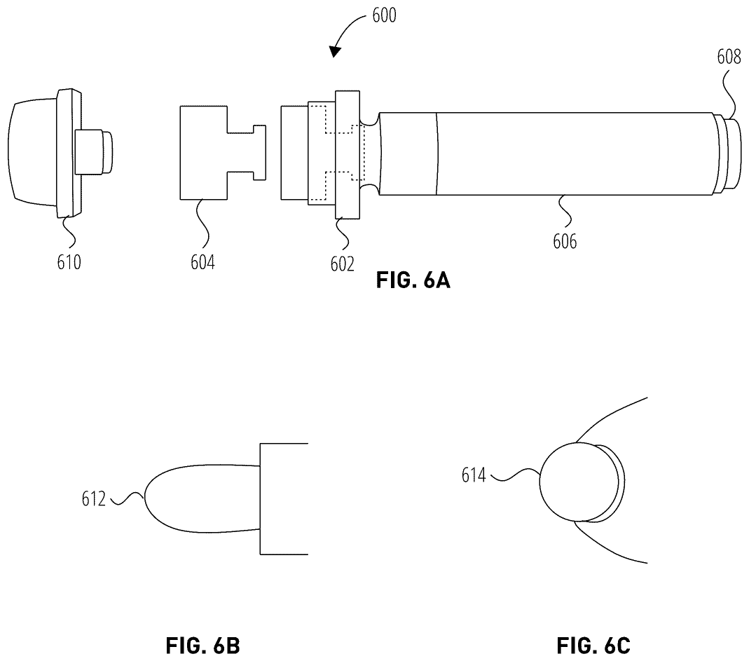

In some embodiments, nibs may be interchangeable. FIG. 6A is an exploded side view of a liquid applicator device 600 in accordance with examples described herein.

The liquid applicator device 600 may include a tip adaptor 602, a nib 604, a liquid container 606, a tail 608, and a cap 610. The nib 604 may include a rectangularly shaped surface configured to be in contact with a surface, such as a human skin. The liquid container 606 may store a liquid, such as a sunscreen mixture. Detailed description of physical, chemical and/or material properties of the liquid container 606, the tail 608 and the sunscreen that have been previously described referring to the liquid container 210, the tail 208 and the sunscreen of FIG. 2, therefore not repeated herein for brevity. In some examples, the cap 610 may be coupled to the nib 604 to prevent evaporation of any ingredients of the sunscreen. In some examples, the nib 604 may have edges that provide different stroke thicknesses. In some examples, the tip adaptor 602 may receive different shapes of nibs and connect such nibs to the liquid container 606. Accordingly, the nib 604 may be removable and replaced with different nibs 604 according to a user's wishes and application goals.

In some examples, the tip may include a nib having a first edge and a second edge shorter than the first edge. In some examples, the nib has a uniform radius that provides a uniform stroke thickness regardless of an angle and a direction of a stroke. In some examples, the tip may include a ballpoint to prevent hardening of the liquid at the tip.

FIG. 6B is a side view of a nib 612 of a liquid applicator device in accordance with examples described herein. In place of the nib 604, the nib 612 may be coupled to the tip adaptor 602. In some examples, the nib 612 may have a conical shape with a uniform radius. Thus, the nib 612 may provide a uniform stroke thickness regardless of an angle and a direction of a stroke.

FIG. 6C is a perspective view of a nib 614 of a liquid applicator device in accordance with examples described herein. In place of the nib 604, the nib 614 may be coupled to the tip adaptor 602. In some examples, the nib 612 may include a ballpoint to prevent solidifying or thickening of the liquid at the tip due to evaporation of some material in the liquid.

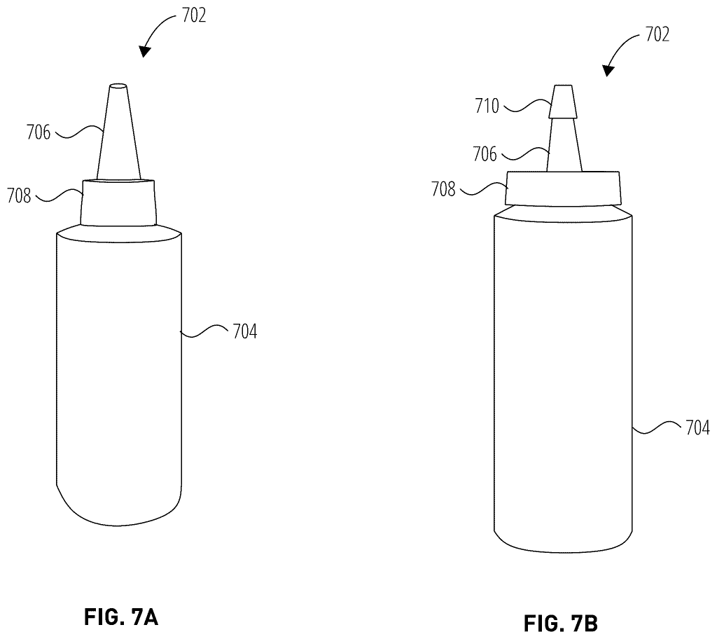

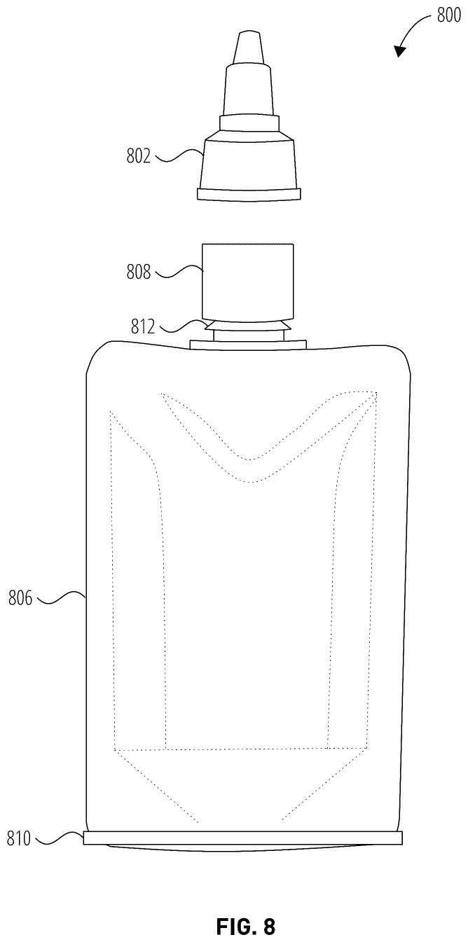

In some examples, a liquid container (e.g., the liquid container 210 of FIG. 2) of a liquid applicator device (e.g., the liquid applicator device 200 of claim 2) may be refillable. Accordingly, after most or all of the liquid inside the liquid container is dispensed, the liquid container may be refilled to enable the liquid applicator devices disclosed herein to continue to be used without wasting the liquid applicator devices. It may, however, be challenging to refill a liquid container of a liquid applicator device with sunscreen mixtures disclosed herein without spilling the sunscreen mixture given their relatively low viscosities, rendering the sunscreen mixtures relatively thin compared to off-the-shelf sunscreen lotions. FIGS. 7A, 7B, and 8 illustrate apparatuses that may be used to refill the liquid container of a liquid applicator device.

FIGS. 7A and 7B are side views of liquid refill devices 702 in accordance with examples described herein. In FIG. 7A, the liquid refill device 702 may include a tip 706 and a tip adaptor 708. In some examples, the tip 706 and the tip adaptor 708 may be integrated as a single component. The liquid refill device 702 may further include a liquid container 704 that is a squeezable container. In some examples, the liquid container 704 may include one or more materials having physical and chemical properties, such as elasticity, flexibility, toughness and corrosion resistance, suitable to contain liquid, such as sunscreen mixture described herein within the liquid container 704. For example, such materials may include, at least in part, low density polyethylene (LDPE), thermoplastic polyurethane (TPU), polyurethane, polyethylene, polypropylene (PP), silicone, etc.

The tip 706 may be tapered toward an end thereof to enable the end of the tip 706 to be inserted into a liquid container (e.g., the liquid container 210 of FIG. 2) of a liquid applicator device (e.g., the liquid applicator device 200 of FIG. 2) to fill or refill the liquid container with a sunscreen mixture. By way of non-limiting example, the end of the tip 706 may be inserted into an aperture at the end 220 of the liquid container 210 and the contents of the liquid refill device 702 may be at least partially emptied into the liquid container 210 of the liquid applicator device 200 (e.g., by squeezing the liquid container 704 portion of the liquid refill device 702). In some embodiments, the end of the tip 706 may be inserted into the other end 208 of the liquid container 210 (e.g., the tail 208 may be removed to allow access to the liquid container 210) and the liquid may be emptied into the liquid container 210.

In some examples, the liquid refill device 702 filled with the liquid (e.g., a sunscreen mixture) may be provided (e.g., sold) separately from the liquid applicator device 200 to a user. In some examples, the liquid applicator device 200 may be provided (e.g., sold) with liquid (e.g., sunscreen mixture) already filling the liquid container 210 and the liquid refill device 702 may be used to refill the liquid container 210 when the liquid in the liquid container 210 diminishes or is used up. In some examples, the liquid applicator device 200 may be provided (e.g., sold) without liquid (e.g., sunscreen mixture) within the liquid container 210 and the liquid refill device 702 may be used to fill the liquid container 210. In some examples, the liquid refill device 702 may be disposable after the liquid (e.g., sunscreen mixture) is at least substantially emptied from the liquid container 704.

In FIG. 7B, the liquid refill device 702 may further include a lid 710 that may cover the tip 706 to prevent leak or any other type of deterioration of the liquid described herein. In some examples, the lid 710 may be a cap that covers an opening of the tip 706 to securely seal the liquid within the liquid refill device 702 while the liquid refill device 702 is not in use.

FIG. 8 is a side view of a liquid refill device 800 in accordance with examples described herein. In FIG. 8, a liquid refill device may include a tip 802 and a liquid container 806 that may be coupled to the tip 802 while in use. In some examples, the liquid container 806 may be a squeezable bag or a pouch. In some examples, the liquid container 806 may include one or more materials having physical and chemical properties, such as elasticity, flexibility, toughness and corrosion resistance, suitable to contain liquid, such as sunscreen mixture described herein within the liquid container 806. For example, such materials may include, at least in part, low density polyethylene (LDPE), thermoplastic polyurethane (TPU), polyurethane, polyethylene, polypropylene (PP), silicone, etc. In some examples, the liquid container 806 may be coupled to a resealable lid 808 while not in use. The resealable lid 808 may be coupled to the liquid container 806 to cover an opening 812 of the liquid container 806 to prevent leak or any other type of deterioration of the liquid described herein.

Similar to the tip 706 of FIGS. 7A and 7B, the tip 802 may be tapered toward an end thereof to enable the end of the tip 802 to be inserted into a liquid container (e.g., the liquid container 210 of FIG. 2) of a liquid applicator device (e.g., the liquid applicator device 200 of FIG. 2) to fill or refill the liquid container with a sunscreen mixture. By way of non-limiting example, the end of the tip 802 may be inserted into an aperture at the end 220 of the liquid container 210 and the contents of the liquid refill device 800 may be at least partially emptied into the liquid container 210 of the liquid applicator device 200 (e.g., by squeezing the liquid container 806 portion of the liquid refill device 800). In some embodiments, the end of the tip 802 may be inserted into the other end 208 of the liquid container 210 (e.g., the tail 208 may be removed to allow access to the liquid container 210) and the liquid may be emptied into the liquid container 210.

In some examples, the liquid refill device 800 filled with the liquid (e.g., a sunscreen mixture) may be provided (e.g., sold) separately from the liquid applicator device 200 to a user. In some examples, the liquid applicator device 200 may be provided (e.g., sold) with liquid (e.g., sunscreen mixture) already filling the liquid container 210 and the liquid refill device 800 may be used to refill the liquid container 210 when the liquid in the liquid container 210 diminishes or is used up. In some examples, the liquid applicator device 200 may be provided (e.g., sold) without liquid (e.g., sunscreen mixture) within the liquid container 210 and the liquid refill device 800 may be used to fill the liquid container 210. In some examples, the liquid refill device 800 may be disposable after the liquid (e.g., sunscreen mixture) is at least substantially emptied from the liquid container 806. In some embodiments, a volume of the liquid container 806 may be selected to be at least substantially the same as a volume of the liquid container 210 of the liquid applicator device 200 to enable a full refill of the liquid container 210 by the liquid refill device 800. In some embodiments, the volume of the liquid container 806 may be selected to be a positive integer multiple of the volume of the liquid container 210 to enable a positive integer number of refills of the liquid container 210 by the refill device 800.

In some examples, the liquid container 806 may be foldable, and/or disposable. In some examples, the liquid container 806 may be provided with a rolling core 810. By rolling the rolling core 810 from a bottom of the liquid container 806 towards the opening 812, the liquid within the liquid container 806 may be dispensed without being wasted by remaining in the liquid container 806.

Accordingly, apparatuses and methods for sunscreen application may be provided in accordance with examples described herein. From the foregoing it will be appreciated that, although specific embodiments of the disclosure have been described herein for purposes of illustration, various modifications may be made without deviating from the spirit and scope of the disclosure.

The particulars shown herein are by way of example and for purposes of illustrative discussion of the preferred embodiments of the present disclosure.

Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural and singular number, respectively. Additionally, the words “herein,” “above,” and “below” and words of similar import, when used in this application, shall refer to this application as a whole and not to any particular portions of the application.

Examples described herein may refer to various components as “coupled” or being “provided to” or “received from” certain components. It is to be understood that in some examples the components are directly coupled one to another, while in other examples the components are coupled with intervening components disposed between them.

Of course, it is to be appreciated that any one of the examples, embodiments, or processes described herein may be combined with one or more other examples, embodiments, and/or processes or be separated and/or performed among separate devices or device portions in accordance with the present devices, and methods.

Finally, the above discussion is intended to be merely illustrative of the present method and device and should not be construed as limiting the appended claims to any particular embodiment or group of embodiments. Thus, while the present method and device have been described in particular detail with reference to exemplary embodiments, it should also be appreciated that numerous modifications and alternative embodiments may be devised by those having ordinary skill in the art without departing from the broader and intended spirit and scope of the present method and device as set forth in the claims that follow. Accordingly, the specification and drawings are to be regarded in an illustrative manner and are not intended to limit the scope of the appended claims.

What is claimed is:

1. A method of providing a sunscreen mixture to a liquid container of a liquid applicator device, the method comprising:
mixing a thinning agent with a pigment agent to produce a pigmented thinning agent;
mixing the pigmented thinning agent with a sunscreen material having a viscosity greater than 500 centipoise (cP) to produce the sunscreen mixture having a viscosity of less than or equal to 100 cP; and
providing the sunscreen mixture to the liquid container of the liquid applicator device.

2. The method of claim 1, wherein the viscosity of the sunscreen mixture is greater than 10 cP.

3. The method of claim 1, wherein the viscosity of the sunscreen mixture is greater than 20 cP.

4. The method of claim 1, wherein the viscosity of the sunscreen mixture is greater than 30 cP.

5. The method of claim 1, wherein mixing the thinning agent with the pigment agent to produce the pigmented thinning agent comprises mixing the thinning agent with titanium dioxide (TiO2) to produce the pigmented thinning agent.

6. The method of claim 1, wherein mixing the thinning agent with the pigment agent to produce the pigmented thinning agent comprises mixing a sun protection factor (SPF) oil with the pigment agent to produce the pigmented thinning agent.

7. The method of claim 6, wherein the SPF oil comprises one or more of raspberry seed oil, avocado oil, argan oil, soybean oil, olive oil, shea butter, almond oil, sesame seed oil, macadamia oil, hemp seed oil, jojoba oil, coconut oil, wheat germ oil, cottonseed oil, peanut oil, or sesame oil.

8. The method of claim 1, wherein the sunscreen mixture is at least substantially equal parts, by volume, thinning agent, pigment agent, and sunscreen material.

9. A liquid applicator device comprising:
a liquid container having a first end and a second end;
a sunscreen mixture within the liquid container, the sunscreen mixture including a pigment agent, a thinning agent, and a sunscreen material, the sunscreen mixture having a viscosity of less than or equal to 100 cP;
a tip configured to couple to the first end, the tip configured to release the sunscreen mixture on a surface in contact with the tip,
wherein the tip is configured to provide a first stroke thickness and further configured to provide a second stroke thickness less than the first stroke thickness depending on one or more of an angle or a position of the tip against the surface.

10. The liquid applicator device of claim 9, wherein the tip comprises a nib comprising:
a first edge configured to provide the first stroke thickness; and
a second edge shorter than the first edge, the second edge configured to provide the second stroke thickness.

11. The liquid applicator device of claim 10, wherein the nib further comprises a corner at a first end of the first edge and a second end of the second edge, wherein the corner of the nib is configured to provide a third stroke thickness less than the second stroke thickness.

12. The liquid applicator device of claim 9, wherein a ratio, by volume, of the thinning agent, the pigment agent, and the sunscreen material of the sunscreen mixture is at least substantially 1:1:1.

13. A liquid applicator device comprising:
a liquid container comprising a first end and a second end opposite to the first end, the liquid container configured to store a liquid;
a sunscreen mixture within the liquid container, the sunscreen material including a pigment agent, a thinning agent, and a sunscreen material;
a tip adaptor coupled to the first end of the liquid container; and
a tail configured to couple to the second end of the liquid container and further configured to seal the sunscreen mixture in the liquid container,
wherein the tip adaptor is configured to couple to any nib of a plurality of nibs having different shapes.

14. The liquid applicator device of claim 13, further comprising a porous foam at the first end of the liquid container,
wherein the porous foam is configured to:
absorb the sunscreen mixture in the liquid container; and
release the sunscreen mixture to the nib evenly.

15. The liquid applicator device of claim 14, further comprising a liquid channel having an inner opening and an outer opening configured to be in contact with the porous foam,
wherein a radius of the inner opening is smaller than a radius of the outer opening.

16. The liquid applicator device of claim 13, further comprising one or more balls configured to move in the liquid container to mix the sunscreen mixture within the liquid container.

17. The liquid applicator device of claim 13, wherein the plurality of nibs comprises a nib comprising a first edge and a second edge shorter than the first edge.

18. The liquid applicator device of claim 13, wherein the plurality of nibs comprises a nib having a conical shape with a uniform radius, the nib configured to provide a uniform stroke thickness regardless of an angle of the liquid applicator relative to an application surface and a direction of a stroke.

19. The liquid applicator device of claim 18, wherein the plurality of nibs comprises a nib including a ballpoint.

20. The liquid applicator device of claim 13, wherein a ratio, by volume, of the thinning agent, the pigment agent, and the sunscreen material of the sunscreen mixture is at least substantially 1:1:1.
